(19) United States
(12) Patent Application Publication
(10) Pub. No.: US 2011/0105557 A1
(43) Pub. Date: May 5, 2011

(54) FARNESYL PROTEIN TRANSFERASE INHIBITOR COMBINATIONS WITH ANTIESTROGEN AGENTS

(76) Inventor: David William End, (US)

(21) Appl. No.: 12/986,351

(22) Filed: Jan. 7, 2011

Related U.S. Application Data
(63) Continuation of application No. 12/240,075, filed on Sep. 29, 2008.
Application No. 12/240,075 is a continuation of application No. 10/468,162, filed on Aug. 14, 2003, now abandoned.
(60) Provisional application No. 60/268,839, filed on Feb. 15, 2001.

(51) Int. Cl. A61K 31/4709; A61P 35/00

(52) U.S. Cl. 514/312

(57) ABSTRACT

The present invention is concerned with combinations of a farnesyl transferase inhibitor and an antiestrogen agent for inhibiting the growth of tumor cells, useful in the treatment of cancer.

The present invention is concerned with combinations of a farnesyl transferase inhibitor and an antiestrogen agent for inhibiting the growth of tumor cells, and useful in the treatment of cancer.

Oncogenes frequently encode protein components of signal transduction pathways which lead to stimulation of cell growth and mitogenesis. Oncogene expression in cultured cells leads to cellular transformation, characterized by the ability of cells to grow in soft agar and the growth of cells as dense foci lacking the contact inhibition exhibited by non-transformed cells. Mutation and/or overexpression of certain oncogenes is frequently associated with human cancer. A particular group of oncogenes is known as ras which have been identified in mammals, birds, insects, mollusks, plants, fungi and yeasts. The family of mammalian ras oncogenes consists of three major members (“isoforms”): H-ras, K-ras and N-ras oncogenes. These ras oncogenes code for highly related proteins generically known as p21^{ras}. Once attached to plasma membranes, the mutant or oncogenic forms of p21^{ras} will provide a signal for the transformation and uncontrolled growth of malignant tumor cells. To acquire this transforming potential, the precursor of the p21^{ras} oncoprotein must undergo an enzymatically catalyzed farnesylation of the cysteine residue located in a carboxyl-terminal tetrapeptide. Therefore, inhibitors of the enzyme that catalyzes this modification, farnesyl protein transferase, will prevent the membrane attachment of p21^{ras} and block the aberrant growth of ras-transformed tumors. Hence, it is generally accepted in the art that farnesyl transferase inhibitors can be very useful as anticancer agents for tumors in which ras contributes to transformation.

Since mutated, oncogenic forms of ras are frequently found in many human cancers, most notably in more than 50% of colon and pancreatic carcinomas (Kohl et al., Science, vol 260, 1834-1837, 1993), it has been suggested that farnesyl tranferase inhibitors can be very useful against these types of cancer. Following further investigations, it has been found that a farnesyl transferase inhibitor is capable of demonstrating antiproliferative effects in vitro and antitumor effects in vivo in a variety of human tumor cell lines with and without ras gene mutations.

WO-97/21701 describes the preparation, formulation and pharmaceutical properties of farnesyl protein transferase inhibiting (imidazoly-5-yl)methyl-2-quinolinone derivatives of formulas (I), (II) and (III), as well as intermediates of formula (II) and (III) that are metabolized in vivo to the compounds of formula (I). The compounds of formulas (I), (II) and (III) are represented by

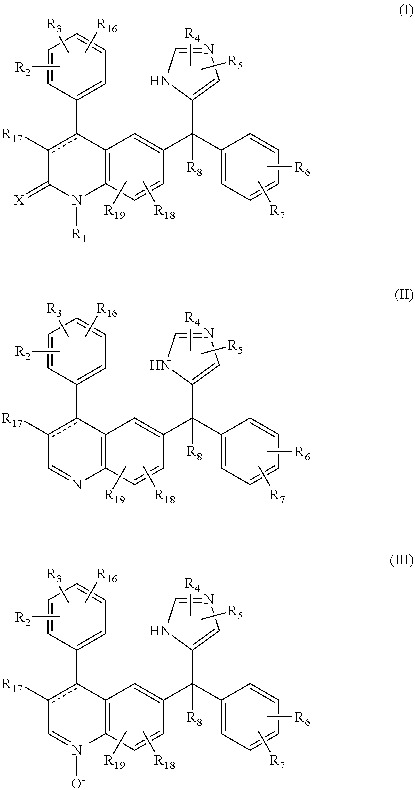

the pharmaceutically acceptable acid or base addition salts and the stereochemically isomeric forms thereof, wherein
the dotted line represents an optional bond;
X is oxygen or sulfur;
R¹ is hydrogen, C₁₋₁₂alkyl, Ar¹, Ar²C₁₋₆alkyl, quinolinylC₁₋₆alkyl, pyridylC₁₋₆alkyl, hydroxyC₁₋₆alkyl, C₁₋₆alkyloxyC₁₋₆alkyl, mono- or di(C₁₋₆alkyl)aminoC₁₋₆alkyl, aminoC₁₋₆alkyl,
   or a radical of formula -Alk¹-C(═O)—R⁹, -Alk¹-S(O)—R⁹ or -Alk¹-S(O)₂—R⁹,
   wherein Alk¹ is C₁₋₆alkanediyl,
      R⁹ is hydroxy, C₁₋₆alkyl, C₁₋₆alkyloxy, amino, C₁₋₈alkylamino or C₁₋₈alkylamino substituted with C₁₋₆alkyloxycarbonyl;
R², R³ and R¹⁶ each independently are hydrogen, hydroxy, halo, cyano, C₁₋₆alkyl, C₁₋₆alkyloxy, hydroxyC₁₋₆alkyloxy, C₁₋₆alkyloxyC₁₋₆alkyloxy, aminoC₁₋₆alkyloxy, mono- or di(C₁₋₆alkyl)aminoC₁₋₆alkyloxy, Ar¹, Ar²C₁₋₆alkyl, Ar²oxy, Ar²C₁₋₆alkyloxy, hydroxycarbonyl, C₁₋₆alkyloxycarbonyl, trihalomethyl, trihalomethoxy, C₂₋₆alkenyl, 4,4-dimethyloxazolyl; or
   when on adjacent positions R² and R³ taken together may form a bivalent radical of formula

—O—CH₂—O— (a-1),

—O—CH₂—CH₂—O— (a-2),

—O—CH═CH— (a-3),

—O—CH₂—CH₂— (a-4),

—O—CH₂—CH₂—CH₂— (a-5), or

—CH═CH—CH═CH— (a-6);
R⁴ and R⁵ each independently are hydrogen, halo, Ar¹, C₁₋₆alkyl, hydroxyC₁₋₆alkyl, C₁₋₆alkyloxyC₁₋₆alkyl, C₁₋₆alkyloxy, C₁₋₆alkylthio, amino, hydroxycarbonyl, C₁₋₆alkyloxycarbonyl, C₁₋₆alkylS(O)C₁₋₆alkyl or C₁₋₆alkylS(O)₂C₁₋₆alkyl;
R⁶ and R⁷ each independently are hydrogen, halo, cyano, Cl₁₋₆alkyl, C₁₋₆alkyloxy, Ar²oxy, trihalomethyl, C₁₋₆alkylthio, di(C₁₋₆alkyl)amino, or
   when on adjacent positions R⁶ and R⁷ taken together may form a bivalent radical of formula

—O—CH₂—O— (c-1), or

—CH═CH—CH═CH— (c-2);
R⁸ is hydrogen, C₁₋₆alkyl, cyano, hydroxycarbonyl, C₁₋₆alkyloxycarbonyl, C₁₋₆alkylcarbonylC₁₋₆alkyl, cyanoC₁₋₆alkyl, C₁₋₆alkyloxycarbonylC₁₋₆alkyl, carboxyC₁₋₆alkyl, hydroxyC₁₋₆alkyl, aminoC₁₋₆alkyl, mono- or di(C₁₋₆alkyl)-aminoC₁₋₆alkyl, imidazolyl, haloC₁₋₆alkyl, C₁₋₆alkyloxyC₁₋₆alkyl, aminocarbonylC₁₋₆alkyl, or a radical of formula

—O—R¹⁰ (b-1),

—S—R¹⁰ (b-2),

—N—R¹¹R¹² (b-3),
wherein R¹⁰ is hydrogen, C₁₋₆alkyl, C₁₋₆alkylcarbonyl, Ar¹, Ar²C₁₋₆alkyl, C₁₋₆alkyloxycarbonylC₁₋₆alkyl, or a radical or formula -Alk²-OR¹³ or -Alk²-NR¹⁴R¹⁵;
   R¹¹ is hydrogen, C₁₋₁₂alkyl, Ar¹ or Ar²C₁₋₆alkyl;
   R¹² is hydrogen, C₁₋₆alkyl, C₁₋₁₆alkylcarbonyl, C₁₋₆alkyloxycarbonyl, C₁₋₆alkylaminocarbonyl, Ar¹, Ar²C₁₋₆alkyl, C₁₋₆alkylcarbonyl-C₁₋₆alkyl, a natural amino acid, Ar¹carbonyl, Ar²C₁₋₆alkylcarbonyl, aminocarbonylcarbonyl, C₁₋₆alkyloxyC₁₋₆alkylcarbonyl, hydroxy, C₁₋₆alkyloxy, aminocarbonyl, di(C₁₋₆alkyl)aminoC₁₋₆alkylcarbonyl, amino, C₁₋₆alkylamino, C₁₋₆alkylcarbonylamino, or a radical or formula -Alk²-OR¹³ or -Alk²-NR¹⁴R¹⁵;
wherein Alk² is C₁₋₆alkanediyl;
   R¹³ is hydrogen, C₁₋₆alkyl, C₁₋₆alkylcarbonyl, hydroxy-C₁₋₆alkyl, Ar¹ or Ar²C₁₋₆alkyl;
   R¹⁴ is hydrogen, C₁₋₆alkyl, Ar¹or Ar²C₁₋₆alkyl;
   R¹⁵ is hydrogen, C₁₋₆alkyl, C₁₋₆alkylcarbonyl, Ar¹ or Ar²C₁₋₆alkyl;
R¹⁷ is hydrogen, halo, cyano, C₁₋₆alkyl, C₁₋₆alkyloxycarbonyl, Ar¹;
R¹⁸ is hydrogen, C₁₋₆alkyl, C₁₋₆alkyloxy or halo;
R¹⁹ is hydrogen or C₁₋₆alkyl;
Ar¹ is phenyl or phenyl substituted with C₁₋₆alkyl, hydroxy, amino, C₁₋₆alkyloxy or halo; and
Ar² is phenyl or phenyl substituted with C₁₋₆alkyl, hydroxy, amino, C₁₋₆alkyloxy or halo.

WO-97/16443 concerns the preparation, formulation and pharmaceutical properties of farnesyl protein transferase inhibiting compounds of formula (IV), as well as intermediates of formula (V) and (VI) that are metabolized in vivo to the compounds of formula (IV). The compounds of formulas (IV), (V) and (VI) are represented by

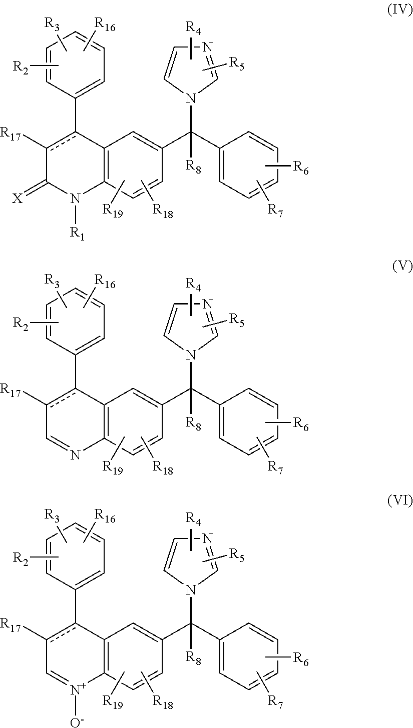

the pharmaceutically acceptable acid or base addition salts and the stereochemically isomeric forms thereof, wherein
the dotted line represents an optional bond;
X is oxygen or sulfur;
R¹ is hydrogen, C₁₋₁₂alkyl, Ar¹, Ar²C₁₋₆alkyl, quinolinylC₁₋₆alkyl, pyridyl-C₁₋₆alkyl, hydroxyC₁₋₆alkyl, C₁₋₆alkyloxyC₁₋₆alkyl, mono- or di(C₁₋₆alkyl)aminoC₁₋₆alkyl, aminoC₁₋₆alkyl,
   or a radical of formula -Alk¹-C(═O)—R⁹, -Alk¹-S(O)—R⁹ or -Alk¹-S(O)₂—R⁹,
   wherein Alk¹ is C₁₋₆alkanediyl,
      R⁹ is hydroxy, C₁₋₆alkyl, C₁₋₆alkyloxy, amino, C₁₋₈alkylamino or C₁₋₈alkylamino substituted with C₁₋₆alkyloxycarbonyl;
R² and R³ each independently are hydrogen, hydroxy, halo, cyano, C₁₋₆alkyl, C₁₋₆alkyloxy, hydroxyC₁₋₆alkyloxy, C₁₋₆alkyloxyC₁₋₆alkyloxy, amino-C₁₋₆alkyloxy, mono- or di(C₁₋₆alkyl)aminoC₁₋₆alkyloxy, Ar¹, Ar²C₁₋₆alkyl, Ar²oxy, Ar²C₁₋₆alkyloxy, hydroxycarbonyl, C₁₋₆alkyloxycarbonyl, trihalomethyl, trihalomethoxy, C₂₋₆alkenyl; or
   when on adjacent positions R² and R³ taken together may form a bivalent radical of formula

—O—CH₂—O— (a-1),

—O—CH₂—CH₂—O— (a-2),

—O—CH═CH— (a-3),

—O—CH₂—CH₂— (a-4),

—O—CH₂—CH₂—CH₂— (a-5), or

—CH═CH—CH═CH— (a-6);
R⁴ and R⁵ each independently are hydrogen, Ar¹, C₁₋₆alkyl, C₁₋₆alkyloxyC₁₋₆alkyl, C₁₋₆alkyloxy, C₁₋₆alkylthio, amino, hydroxycarbonyl, C₁₋₆alkyloxycarbonyl, C₁₋₆alkylS(O)C₁₋₆alkyl or C₁₋₆alkylS(O)₂C₁₋₆alkyl;
R⁶ and R⁷ each independently are hydrogen, halo, cyano, C₁₋₆alkyl, C₁₋₆alkyloxy or Ar²oxy;
R⁸ is hydrogen, C₁₋₆alkyl, cyano, hydroxycarbonyl, C₁₋₆alkyloxycarbonyl, C₁₋₆alkylcarbonylC₁₋₆alkyl, cyanoC₁₋₆alkyl, C₁₋₆alkyloxycarbonylC₁₋₆alkyl, hydroxycarbonylC₁₋₆alkyl, hydroxyC₁₋₆alkyl, aminoC₁₋₆alkyl, mono- or di(C₁₋₆alkyl)aminoC₁₋₆alkyl, haloC₁₋₆alkyl, C₁₋₆alkyloxyC₁₋₆alkyl, aminocarbonylC₁₋₆alkyl, Ar¹, Ar²C₁₋₆alkyloxyC₁₋₆alkyl, C₁₋₆alkylthioC₁₋₆alkyl;
R¹⁰ is hydrogen, C₁₋₆alkyl, C₁₋₆alkyloxy or halo;
R¹¹ is hydrogen or C₁₋₆alkyl;
Ar¹ is phenyl or phenyl substituted with C₁₋₆alkyl, hydroxy, amino, C₁₋₆alkyloxy or halo;
Ar² is phenyl or phenyl substituted with C₁₋₆alkyl, hydroxy, amino, C₁₋₆alkyloxy or halo.

WO-98/40383 concerns the preparation, formulation and pharmaceutical properties of farnesyl protein transferase inhibiting compounds of formula (VII)

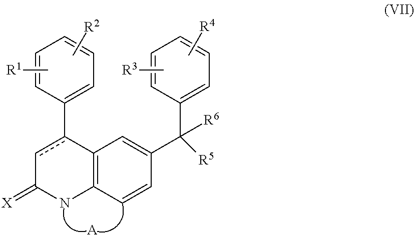

the pharmaceutically acceptable acid addition salts and the stereochemically isomeric forms thereof, wherein
the dotted line represents an optional bond;
X is oxygen or sulfur;
-A- is a bivalent radical of formula
   —CH═CH— (a-1),
   —CH₂—CH₂— (a-2),
   —CH₂—CH₂— CH₂— (a-3),
   —CH₂—O— (a-4),
   —CH₂—CH₂—O— (a-5),
   —CH₂—S— (a-6),
   —CH₂—CH₂—S— (a-7),
   —CH═N— (a-8),
   —N═N— (a-9), or
   —CO—NH— (a-10);
   wherein optionally one hydrogen atom may be replaced by C₁₋₄alkyl or Ar¹;
R¹ and R² each independently are hydrogen, hydroxy, halo, cyano, C₁₋₆alkyl, trihalomethyl, trihalomethoxy, C₂₋₆alkenyl, C₁₋₆alkyloxy, hydroxyC₁₋₆alkyloxy, C₁₋₆alkyloxyC₁₋₆alkyloxy, C₁₋₆alkyloxycarbonyl, aminoC₁₋₆alkyloxy, mono- or di(C₁₋₆alkyl)aminoC₁₋₆alkyloxy, Ar², Ar²—C₁₋₆alkyl, Ar²-oxy, Ar²—C₁₋₆alkyloxy; or when on adjacent positions R¹ and R² taken together may form a bivalent radical of formula

—O—CH₂—O— (b-1),

—O—CH₂—CH₂—O— (b-2),

—O—CH═CH— (b-3),

—O—CH₂—CH₂— (b-4),

—O—CH₂—CH₂—CH₂— (b-5), or

—CH═CH—CH═CH— (b-6);
R³ and R⁴ each independently are hydrogen, halo, cyano, C₁₋₆alkyl, C₁₋₆alkyloxy, Ar³-oxy, C₁₋₆alkylthio, di(C₁₋₆alkyl)amino, trihalomethyl, trihalomethoxy, or when on adjacent positions R³ and R⁴ taken together may form a bivalent radical of formula

—O—CH₂—O— (c-1),

—O—CH₂—CH₂—O— (c-2), or

—CH═CH—CH═CH— (c-3);
R⁵ is a radical of formula

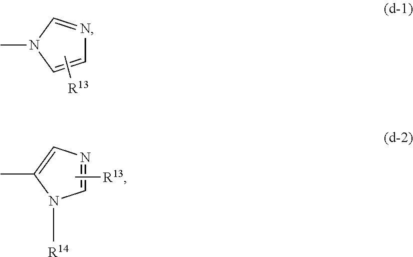

wherein R¹³ is hydrogen, halo, Ar⁴, C₁₋₆alkyl, hydroxyC₁₋₆alkyl, C₁₋₆alkyloxy-C₁₋₆alkyl, C₁₋₆alkyloxy, C₁₋₆alkylthio, amino, C₁₋₆alkyloxycarbonyl, C₁₋₆alkylS(O)C₁₋₆alkyl or C₁₋₆alkylS(O)₂C₁₋₆alkyl;
   R¹⁴ is hydrogen, C₁₋₆alkyl or di(C₁₋₄alkyl)aminosulfonyl;
R⁶ is hydrogen, hydroxy, halo, C₁₋₆alkyl, cyano, haloC₁₋₆alkyl, hydroxyC₁₋₆alkyl, cyanoC₁₋₆alkyl, aminoC₁₋₆alkyl, C₁₋₆alkyloxyC₁₋₆alkyl, C₁₋₆alkylthio C₁₋₆alkyl, aminocarbonylC₁₋₆alkyl, C₁₋₆alkyloxycarbonylC₁₋₆alkyl, C₁₋₆alkylcarbonyl-C₁₋₆alkyl, C₁₋₆alkyloxycarbonyl, mono- or di(C₁₋₆alkyl)aminoC₁₋₆alkyl, Ar⁵, Ar⁵—C₁₋₆alkyloxyC₁₋₆alkyl; or a radical of formula

—O—R⁷ (e-1),

—S—R⁷ (e-2),

—N—R⁸R⁹ (e-3),
wherein R⁷ is hydrogen, C₁₋₆alkyl, C₁₋₆alkylcarbonyl, Ar⁶, Ar⁶—C₁₋₆alkyl, C₁₋₆alkyloxycarbonylC₁₋₆alkyl, or a radical of formula -Alk-OR¹⁰ or -Alk-NR¹¹R¹²;
   R⁸ is hydrogen, C₁₋₆alkyl, Ar⁷ or Ar⁷—C₁₋₆alkyl;
   R⁹ is hydrogen, C₁₋₆alkyl, C₁₋₆alkylcarbonyl, C₁₋₆alkyloxycarbonyl, C₁₋₆alkylaminocarbonyl, Ar⁸, Ar⁸—C₁₋₆alkyl, C₁₋₆alkylcarbonyl-C₁₋₆alkyl, Ar⁸-carbonyl, Ar⁸—C₁₋₆alkylcarbonyl, aminocarbonyl-carbonyl, C₁₋₆alkyloxyC₁₋₆alkylcarbonyl, hydroxy, C₁₋₆alkyloxy, aminocarbonyl, di(C₁₋₆alkyl)aminoC₁₋₆alkylcarbonyl, amino, C₁₋₆alkylamino, C₁₋₆alkylcarbonylamino,
      or a radical or formula -Alk-OR¹⁰ or -Alk-NR¹¹R¹²;
wherein Alk is C₁₋₆alkanediyl;
   R¹⁰ is hydrogen, C₁₋₆alkyl, C₁₋₆alkylcarbonyl, hydroxy-C₁₋₆alkyl, Arg or Ar⁹—C₁₋₆alkyl;
   R¹¹ is hydrogen, C₁₋₆alkyl, C₁₋₆alkylcarbonyl, Ar¹⁰ or Ar¹⁰—C₁₋₆alkyl;
   R¹² is hydrogen, C₁₋₆alkyl, Ar¹¹ or Ar¹¹—C₁₋₆alkyl; and
Ar¹ to Ar¹¹ are each independently selected from phenyl; or phenyl substituted with halo, C₁₋₆alkyl, C₁₋₆alkyloxy or trifluoromethyl.

WO-98/49157 concerns the preparation, formulation and pharmaceutical properties of farnesyl protein transferase inhibiting compounds of formula (VIII)

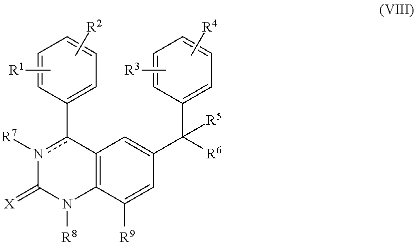

the pharmaceutically acceptable acid addition salts and the stereochemically isomeric forms thereof, wherein
the dotted line represents an optional bond;
X is oxygen or sulfur;
R¹ and R² each independently are hydrogen, hydroxy, halo, cyano, C₁₋₆alkyl, trihalomethyl, trihalomethoxy, C₂₋₆alkenyl, C₁₋₆alkyloxy, hydroxyC₁₋₆alkyloxy, C₁₋₆alkyloxyC₁₋₆alkyloxy, C₁₋₆alkyloxycarbonyl, aminoC₁₋₆alkyloxy, mono- or di(C₁₋₆alkyl)aminoC₁₋₆alkyloxy, Ar¹, Ar¹C₁₋₆alkyl, Ar¹oxy or Ar¹C₁₋₆alkyloxy;
R³ and R⁴ each independently are hydrogen, halo, cyano, C₁₋₆alkyl, C₁₋₆alkyloxy, Ar¹oxy, C₁₋₆alkylthio, di(C₁₋₆alkyl)amino, trihalomethyl or trihalomethoxy;
R⁵ is hydrogen, halo, C₁₋₆alkyl, cyano, haloC₁₋₆alkyl, hydroxyC₁₋₆alkyl, cyanoC₁₋₆alkyl, aminoC₁₋₆alkyl, C₁₋₆alkyloxyC₁₋₆alkyl, C₁₋₆alkylthioC₁₋₆alkyl, aminocarbonylC₁₋₆alkyl, C₁₋₆alkyloxycarbonylC₁₋₆alkyl, C₁₋₆alkylcarbonyl-C₁₋₆alkyl, C₁₋₆alkyloxycarbonyl, mono- or di(C₁₋₆alkyl)aminoC₁₋₆alkyl, Ar¹, Ar¹C₁₋₆alkyloxyC₁₋₆alkyl; or a radical of formula

—O—R¹⁰ (a-1),

—S—R¹⁰ (a-2),

—N—R¹¹R¹² (a-3),
wherein R¹⁰ is hydrogen, C₁₋₆alkyl, C₁₋₆alkylcarbonyl, Ar¹, Ar¹C₁₋₆alkyl, C₁₋₆alkyloxycarbonylC₁₋₆alkyl, or a radical of formula -Alk-OR¹³ or -Alk-NR¹⁴R¹⁵;
   R¹¹ is hydrogen, C₁₋₆alkyl, Ar¹, or Ar¹C₁₋₆alkyl;
   R¹² is hydrogen, C₁₋₆alkyl, C₁₋₆alkylcarbonyl, C₁₋₆alkyloxycarbonyl, C₁₋₆alkylaminocarbonyl, Ar¹, Ar¹C₁₋₆alkyl, C₁₋₆alkylcarbonyl-C₁₋₆ alkyl, Ar¹-carbonyl, Ar¹C₁₋₆alkylcarbonyl, aminocarbonyl-carbonyl, C₁₋₆alkyloxyC₁₋₆alkylcarbonyl, hydroxy, C₁₋₆alkyloxy, aminocarbonyl, di(C₁₋₆alkyl)aminoC₁₋₆alkylcarbonyl, amino, C₁₋₆alkylamino, C₁₋₆alkylcarbonylamino,
      or a radical or formula -Alk-OR¹³ or -Alk-NR¹⁴R¹⁵;
      wherein Alk is C₁₋₆alkanediyl;
         R¹³ is hydrogen, C₁₋₆alkyl, C₁₋₆alkylcarbonyl, hydroxy-C₁₋₆alkyl, Ar¹ or Ar¹C₁₋₆alkyl;
         R¹⁴ is hydrogen, C₁₋₆alkyl, Ar¹ or Ar¹C₁₋₆alkyl;
         R¹⁵ is hydrogen, C₁₋₆alkyl, C₁₋₆alkylcarbonyl, Ar¹ or Ar¹C₁₋₆alkyl;
R⁶ is a radical of formula

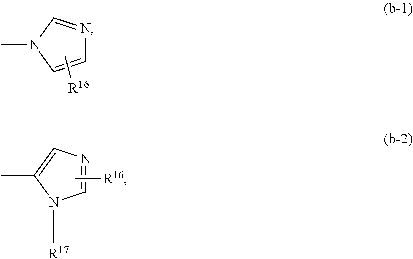

wherein R¹⁶ is hydrogen, halo, Ar¹, C₁₋₆alkyl, hydroxyC₁₋₆alkyl, C₁₋₆alkyloxy-C₁₋₆alkyl, C₁₋₆alkyloxy, C₁₋₆alkylthio, amino, C₁₋₆alkyloxycarbonyl, C₁₋₆alkylthioC₁₋₆alkyl, C₁₋₆alkylS(O)C₁₋₆alkyl or C₁₋₆alkylS(O)₂C₁₋₆alkyl;
   R¹⁷ is hydrogen, C₁₋₆alkyl or di(C₁₋₄alkyl)aminosulfonyl;
R⁷ is hydrogen or C₁₋₆alkyl provided that the dotted line does not represent a bond;
R⁸ is hydrogen, C₁₋₆alkyl or Ar²CH₂ or Het¹CH₂;
R⁹ is hydrogen, C₁₋₆alkyl, C₁₋₆alkyloxy or halo; or
R⁸ and R⁹ taken together to form a bivalent radical of formula

—CH═CH— (c-1),

—CH₂—CH₂— (c-2),

—CH₂—CH₂—CH₂— (c-3),

—CH₂—O— (c-4), or

—CH₂—CH₂—O— (c-5);
Ar¹ is phenyl; or phenyl substituted with 1 or 2 substituents each independently selected from halo, C₁₋₆alkyl, C₁₋₆alkyloxy or trifluoromethyl;
Ar² is phenyl; or phenyl substituted with 1 or 2 substituents each independently selected from halo, C₁₋₆alkyl, C₁₋₆alkyloxy or trifluoromethyl; and
Het¹ is pyridinyl; pyridinyl substituted with 1 or 2 substituents each independently selected from halo, C₁₋₆alkyl, C₁₋₆alkyloxy or trifluoromethyl.

WO-00/39082 concerns the preparation, formulation and pharmaceutical properties of farnesyl protein transferase inhibiting compounds of formula (IX)

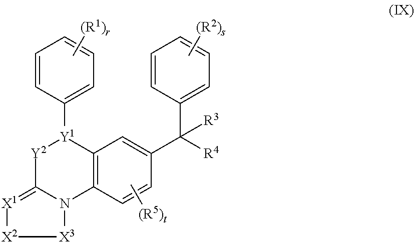

or the pharmaceutically acceptable acid addition salts and the stereochemically isomeric forms thereof, wherein
═X¹—X²—X³— is a trivalent radical of formula
═N—CR⁶═CR⁷— (x-1),
═N—N═CR⁶— (x-2),
═N—NH—C(═O)— (x-3),
═N—N═N— (x-4),
═N—CR⁶═N— (x-5),
═CR⁶—CR⁷═CR⁸— (x-6),
═CR⁶—N═CR⁷— (x-7),
═CR⁶—NH—C(═O)— (x-8), or
═CR⁶—N═N— (x-9);
   wherein each R⁶, R⁷ and R⁸ are independently hydrogen, C₁₋₄alkyl, hydroxy, C₁₋₄alkyloxy, aryloxy, C₁₋₄alkyloxycarbonyl, hydroxyC₁₋₄alkyl, C₁₋₄alkyloxyC₁₋₄alkyl, mono- or di(C₁₋₄alkyl)aminoC₁₋₄alkyl, cyano, amino, thio, C₁₋₄alkylthio, arylthio or aryl;
>Y¹—Y²— is a trivalent radical of formula

>CH—CHR⁹— (y-1),

>C═N— (y-2),

>CH—NR⁹— (y-3), or

>C═CR⁹— (y-4);
wherein each R⁹ independently is hydrogen, halo, halocarbonyl, aminocarbonyl, hydroxyC₁₋₄alkyl, cyano, carboxyl, C₁₋₄alkyl, C₁₋₄alkyloxy, C₁₋₄alkyloxyC₁₋₄alkyl, C₁₋₄alkyloxycarbonyl, mono- or di(C₁₋₄alkyl)amino, mono- or di(C₁₋₄alkyl)aminoC₁₋₄alkyl, aryl;
r and s are each independently 0, 1, 2, 3, 4 or 5;
t is 0, 1, 2 or 3;
each R¹ and R² are independently hydroxy, halo, cyano, C₁₋₆alkyl, trihalomethyl, trihalomethoxy, C₂₋₆alkenyl, C₁₋₆alkyloxy, hydroxyC₁₋₆alkyloxy, C₁₋₆alkylthio, C₁₋₆alkyloxyC₁₋₆alkyloxy, C₁₋₆alkyloxycarbonyl, aminoC₁₋₆alkyloxy, mono- or di(C₁₋₆alkyl)amino, mono- or di(C₁₋₆alkyl)aminoC₁₋₆alkyloxy, aryl, arylC₁₋₆alkyl, aryloxy or arylC₁₋₆alkyloxy, hydroxycarbonyl, C₁₋₆alkyloxycarbonyl, aminocarbonyl, aminoC₁₋₆alkyl, mono- or di(C₁₋₆alkyl)aminocarbonyl, mono- or di(C₁₋₆alkyl)aminoC₁₋₆alkyl; or
two R¹ or R² substituents adjacent to one another on the phenyl ring may independently form together a bivalent radical of formula

—O—CH₂—O— (a-1),

—O—CH₂—CH₂—O— (a-2),

—O═CH═CH— (a-3),

—O—CH₂—CH₂— (a-4),

—O—CH₂—CH₂—CH₂— (a-5), or

—CH═CH—CH═CH— (a-6);
R³ is hydrogen, halo, C₁₋₆alkyl, cyano, haloC₁₋₆alkyl, hydroxyC₁₋₆alkyl, cyanoC₁₋₆alkyl, aminoC₁₋₆alkyl, C₁₋₆alkyloxyC₁₋₆alkyl, C₁₋₆alkylthioC₁₋₆alkyl, aminocarbonylC₁₋₆alkyl, hydroxycarbonyl, hydroxycarbonylC₁₋₆alkyl, C₁₋₆alkyloxycarbonylC₁₋₆alkyl, C₁₋₆alkylcarbonylC₁₋₆alkyl, C₁₋₆alkyloxycarbonyl, aryl, arylC₁₋₆alkyloxyC₁₋₆alkyl, mono- or di(C₁₋₆alkyl)aminoC₁₋₆alkyl;
or a radical of formula

—O—R¹⁰ (b-1),

—S—R¹⁰ (b-2),

—NR¹¹R¹² (b-3),
wherein R¹⁰ is hydrogen, C₁₋₆alkyl, C₁₋₆alkylcarbonyl, aryl, arylC₁₋₆alkyl, C₁₋₆alkyloxycarbonylC₁₋₆alkyl, or a radical of formula -Alk-OR¹³ or -Alk-NR¹⁴R¹⁵;
   R¹¹ is hydrogen, C₁₋₆alkyl, aryl or arylC₁₋₆alkyl;
   R¹² is hydrogen, C₁₋₆alkyl, aryl, hydroxy, amino, C₁₋₆alkyloxy, C₁₋₆alkylcarbonylC₁₋₆alkyl, arylC₁₋₆alkyl, C₁₋₆alkylcarbonylamino, mono- or di(C₁₋₆alkyl)amino, C₁₋₆alkylcarbonyl, aminocarbonyl, arylcarbonyl, haloC₁₋₆alkylcarbonyl, arylC₁₋₆alkylcarbonyl, C₁₋₆alkyloxycarbonyl, C₁₋₆alkyloxyC₁₋₆alkylcarbonyl, mono- or di(C₁₋₆alkyl)aminocarbonyl wherein the alkyl moiety may optionally be substituted by one or more substituents independently selected from aryl or C₁₋₃alkyloxycarbonyl, aminocarbonylcarbonyl, mono- or di(C₁₋₆alkyl)aminoC₁₋₆alkylcarbonyl, or a radical or formula -Alk-OR¹³ or -Alk-NR¹⁴R¹⁵;
   wherein Alk is C₁₋₆alkanediyl;
      R¹³ is hydrogen, C₁₋₆alkyl, C₁₋₆alkylcarbonyl, hydroxyC₁₋₆alkyl, aryl or arylC₁₋₆alkyl;
      R¹⁴ is hydrogen, C₁₋₆alkyl, aryl or arylC₁₋₆alkyl;
      R¹⁵ is hydrogen, C₁₋₆alkyl, C₁₋₆alkylcarbonyl, aryl or arylC₁₋₆alkyl;
R⁴ is a radical of formula

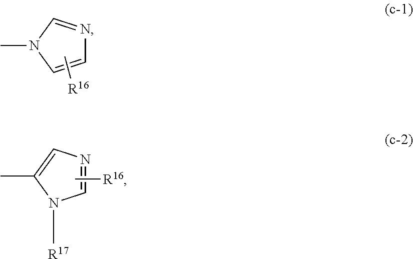

wherein R¹⁶ is hydrogen, halo, aryl, C₁₋₆alkyl, hydroxyC₁₋₆alkyl, C₁₋₆alkyloxyC₁₋₆alkyl, C₁₋₆alkyloxy, C₁₋₆alkylthio, amino, mono- or di(C₁₋₄alkyl)amino, hydroxycarbonyl, C₁₋₆alkyloxycarbonyl, C₁₋₆alkylthioC₁₋₆alkyl, C₁₋₆alkylS(O)C₁₋₆alkyl or C₁₋₆alkylS(O)₂C₁₋₆alkyl;
   R¹⁶ may also be bound to one of the nitrogen atoms in the imidazole ring of formula (c-1) or (c-2), in which case the meaning of R¹⁶ when bound to the nitrogen is limited to hydrogen, aryl, C₁₋₆alkyl, hydroxyC₁₋₆alkyl, C₁₋₆alkyloxyC₁₋₆alkyl, C₁₋₆alkyloxycarbonyl, C₁₋₆alkylS(O)C₁₋₆alkyl or C₁₋₆alkylS(O)₂C₁₋₆alkyl;
   R¹⁷ is hydrogen, C₁₋₆alkyl, C₁₋₆alkyloxyC₁₋₆alkyl, arylC₁₋₆alkyl, trifluoromethyl or di(C₁₋₄alkyl)aminosulfonyl;
R⁵ is C₁₋₆alkyl, C₁₋₆alkyloxy or halo;
aryl is phenyl, naphthalenyl or phenyl substituted with 1 or more substituents each independently selected from halo, C₁₋₆alkyl, C₁₋₆alkyloxy or trifluoromethyl.

Many breast cancers have estrogen receptors and growth of these tumors can be stimulated by estrogen. Antiestrogen agents have therefore been proposed and used for the treatment of cancers especially breast cancer. One of the most widely used of such agents is tamoxifen which is a competitive inhibitor of estradiol binding to the estrogen receptor (ER). When bound to the ER, tamoxifen induces a change in the three-dimensional shape of the receptor, inhibiting its binding to the estrogen responsive element (ERE) on DNA. Under normal physiological conditions, estrogen stimulation increases tumor cell production of transforming growth cell b (TGF-b), an autocrine inhibitor of tumor cell growth. By blocking these pathways, the net effect of tamoxifen treatment is to decrease the autocrine stimulation of breast cancer growth. In addition, tamoxifen decreases the local production of insulin-like growth factor (IGF-1) by surrounding tissues: IGF-1 is a paracrine growth factor for the breast cancer cell (Jordan and Murphy, Endocr. Rev., 1990, 11; 578-610). Tamoxifen is the endocrine treatment of choice for post-menopausal women with metastatic breast cancer or at a high risk of recurrences from the disease. Tamoxifen is also used in pre-menopausal women with ER-positive tumors. There are various potential side-effects of long-term tamoxifen treatment for example the possibility of endometrial cancer and the occurrence of thrombo-embolic events. Thus, although tamoxifen has been widely used as a chemotherapeutic agent in humans, it is not therapeutically effective in all patients or against all types of tumors. Other estrogen receptor antagonists or selective estrogen receptor modulators include toremifene, droloxifene, faslodex and raloxifene.

In postmenopausal women, the principal source of circulating estrogen is from conversion of adrenal and ovarian androgens (androstenedione and testosterone) to estrogens (estrone and estradiol) by the aromatase enzyme in peripheral tissues. Estrogen deprivation through aromatase inhibition or inactivation is an effective and selective treatment for some postmenopausal patients with hormone-dependent breast cancer. Examples of aromatase inhibitors or inactivators include exemestane, anastrozole, letrazole and vorozole.

The term “antiestrogen agent” is used herein to include not only estrogen receptor antagonists and selective estrogen receptor modulators but also aromatase inhibitors as discussed above.

WO-01/45740 describes compositions and methods for treating and/or preventing breast cancer including compositions comprising at least one selective estrogen receptor modulator for example tamoxifen and at least one farnesyl transferase inhibitor for example FTI-277.

There is a need to increase the inhibitory efficacy of antiestrogen agents against tumor growth and also to provide a means for the use of lower dosages of such agents to reduce the potential of adverse toxic side effects to the patient.

It is an object of the invention to provide a therapeutic combination of an antiestrogen agent and a farnesyl transferase inhibitor of the type described above which has an advantageous inhibitory effect against tumor cell growth, in comparison with the respective effects shown by the individual components of the combination.

According to the invention therefore we provide a combination of an antiestrogen agent and a farnesyl transferase inhibitor of formula (I), (II), (III), (IV), (V), (VI), (VII), (VIII) or (IX) above, in particular a compound of formula (I), (II) or (III):

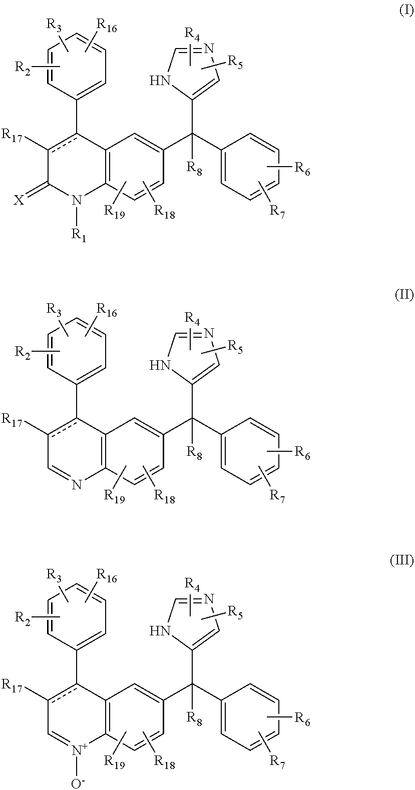

the pharmaceutically acceptable acid or base addition salts and the stereochemically isomeric forms thereof, wherein
the dotted line represents an optional bond;
X is oxygen or sulfur;
R¹ is hydrogen, C₁₋₁₂alkyl, Ar¹, Ar²C₁₋₆alkyl, quinolinylC₁₋₆alkyl, pyridyl-C₁₋₆alkyl, hydroxyC₁₋₆alkyl, C₁₋₆alkyloxyC₁₋₆alkyl, mono- or di(C₁₋₆alkyl)aminoC₁₋₆alkyl, aminoC₁₋₆alkyl,
   or a radical of formula -Alk¹-C(═O)—R⁹, -Alk¹-S(O)—R⁹ or -Alk¹-S(O)₂—R⁹,
   wherein Alk¹ is C₁₋₆alkanediyl,
      R⁹ is hydroxy, C₁₋₆alkyl, C₁₋₆alkyloxy, amino, C₁₋₈alkylamino or C₁₋₈alkylamino substituted with C₁₋₆alkyloxycarbonyl;
R², R³ and R¹⁶ each independently are hydrogen, hydroxy, halo, cyano, C₁₋₆alkyl, C₁₋₆alkyloxy, hydroxyC₁₋₆alkyloxy, C₁₋₆alkyloxyC₁₋₆alkyloxy, aminoC₁₋₆alkyloxy, mono- or di(C₁₋₆alkyl)aminoC₁₋₆alkyloxy, Ar¹, Ar²C₁₋₆alkyl, Ar²oxy, Ar²C₁₋₆alkyloxy, hydroxycarbonyl, C₁₋₆alkyloxycarbonyl, trihalomethyl, trihalomethoxy, C₂₋₆alkenyl, 4,4-dimethyloxazolyl; or
   when on adjacent positions R² and R³ taken together may form a bivalent radical of formula

—O—CH₂—O— (a-1),

—O—CH₂—CH₂—O— (a-2),

—O—CH═CH— (a-3),

—O—CH₂—CH₂— (a-4),

—O—CH₂—CH₂—CH₂— (a-5), or

—CH═CH—CH═CH— (a-6);
R⁴ and R⁵ each independently are hydrogen, halo, Ar¹, C₁₋₆alkyl, hydroxyC₁₋₆alkyl, C₁₋₆alkyloxyC₁₋₆alkyl, C₁₋₆alkyloxy, C₁₋₆alkylthio, amino, hydroxycarbonyl, C₁₋₆alkyloxycarbonyl, C₁₋₆alkylS(O)C₁₋₆alkyl or C₁₋₆alkylS(O)₂C₁₋₆alkyl;
R⁶ and R⁷ each independently are hydrogen, halo, cyano, C₁₋₆alkyl, C₁₋₆alkyloxy, Ar²oxy, trihalomethyl, C₁₋₆alkylthio, di(C₁₋₆alkyl)amino, or
   when on adjacent positions R⁶ and R⁷ taken together may form a bivalent radical of formula

—O—CH₂—O— (c-1), or

—CH═CH—CH═CH— (c-2);
R⁸ is hydrogen, C₁₋₆alkyl, cyano, hydroxycarbonyl, C₁₋₆alkyloxycarbonyl, C₁₋₆alkylcarbonylC₁₋₆alkyl, cyanoC₁₋₆alkyl, C₁₋₆alkyloxycarbonylC₁₋₆alkyl, carboxyC₁₋₆alkyl, hydroxyC₁₋₆alkyl, aminoC₁₋₆alkyl, mono- or di(C₁₋₆alkyl)aminoC₁₋₆alkyl, imidazolyl, haloC₁₋₆alkyl, C₁₋₆alkyloxyC₁₋₆alkyl, aminocarbonylC₁₋₆alkyl, or a radical of formula

—O—R¹⁰ (b-1),

—S—R¹⁰ (b-2),

—N—R¹¹R¹² (b-3),
wherein R¹⁰ is hydrogen, C₁₋₆alkyl, C₁₋₆alkylcarbonyl, Ar¹, Ar²C₁₋₆alkyl, C₁₋₆alkyloxycarbonylC₁₋₆alkyl, or a radical or formula -Alk²-OR¹³ or -Alk²-NR¹⁴R¹⁵;
   R¹¹ is hydrogen, C₁₋₁₂alkyl, Ar¹ or Ar²C₁₋₆alkyl;
   R¹² is hydrogen, C₁₋₆alkyl, C₁₋₁₆alkylcarbonyl, C₁₋₆alkyloxycarbonyl, C₁₋₆alkylaminocarbonyl, Ar¹, Ar²C₁₋₆alkyl, C₁₋₆alkylcarbonyl-C₁₋₆alkyl, a natural amino acid, Ar¹carbonyl, Ar²C₁₋₆alkylcarbonyl, aminocarbonylcarbonyl, C₁₋₆alkyloxyC₁₋₆alkylcarbonyl, hydroxy, C₁₋₆alkyloxy, aminocarbonyl, di(C₁₋₆alkyl)aminoC₁₋₆alkylcarbonyl, amino, C₁₋₆alkylamino, C₁₋₆alkylcarbonylamino,
      or a radical or formula -Alk²-OR¹³ or -Alk²-NR¹⁴R¹⁵;
      wherein Alk² is C₁₋₆alkanediyl;
         R¹³ is hydrogen, C₁₋₆alkyl, C₁₋₆alkylcarbonyl, hydroxy-C₁₋₆alkyl, Ar¹ or Ar²C₁₋₆alkyl;
         R¹⁴ is hydrogen, C₁₋₆alkyl, Ar¹ or Ar²C₁₋₆alkyl;
         R¹⁵ is hydrogen, C₁₋₆alkyl, C₁₋₆alkylcarbonyl, Ar¹ or Ar²C₁₋₆alkyl;
R¹⁷ is hydrogen, halo, cyano, C₁₋₆alkyl, C₁₋₆alkyloxycarbonyl, Ar¹;
R¹⁸ is hydrogen, C₁₋₆alkyl, C₁₋₆alkyloxy or halo;
R¹⁹ is hydrogen or C₁₋₆alkyl;
Ar¹ is phenyl or phenyl substituted with C₁₋₆alkyl, hydroxy, amino, C₁₋₆alkyloxy or halo; and
Ar² is phenyl or phenyl substituted with C₁₋₆alkyl, hydroxy, amino, C₁₋₆alkyloxy or halo.

In Formulas (I), (II) and (III), R⁴ or R⁵ may also be bound to one of the nitrogen atoms in the imidazole ring. In that case the hydrogen on the nitrogen is replaced by R⁴ or R⁵ and the meaning of R⁴ and R⁵ when bound to the nitrogen is limited to hydrogen, Ar¹, C₁₋₆ alkyl, hydroxyC₁₋₆alkyl, C₁₋₆ alkyloxyC₁₋₆alkyl, C₁₋₆ alkyloxycarbonyl, C₁₋₆alkylS(O)C₁₋₆alkyl, C₁₋₆alkylS(O)₂C₁₋₆alkyl.

Preferably the substituent R¹⁸ is situated on the 5 or 7 position of the quinolinone moiety and substituent R¹⁹ is situated on the 8 position when R¹⁸ is on the 7-position.

Interesting compounds are these compounds of formula (I) wherein X is oxygen.

Also interesting compounds are these compounds of formula (I) wherein the dotted line represents a bond, so as to form a double bond.

Another group of interesting compounds are those compounds of formula (I) wherein R¹ is hydrogen, C₁₋₆alkyl, C₁₋₆alkyloxyC₁₋₆alkyl, di(C₁₋₆alkyl)aminoC₁₋₆alkyl, or a radical of formula -Alk¹-C(═O)—R⁹, wherein Alk¹ is methylene and R⁹ is C₁₋₈alkylamino substituted with C₁₋₆alkyloxycarbonyl.

Still another group of interesting compounds are those compounds of formula (I) wherein R³ is hydrogen or halo; and R² is halo, C₁₋₆alkyl, C₂₋₆alkenyl, C₁₋₆alkyloxy, trihalomethoxy or hydroxyC₁₋₆alkyloxy.

A further group of interesting compounds are those compounds of formula (I) wherein R² and R³ are on adjacent positions and taken together to form a bivalent radical of formula (a-1), (a-2) or (a-3).

A still further group of interesting compounds are those compounds of formula (I) wherein R⁵ is hydrogen and R⁴ is hydrogen or C₁₋₆alkyl.

Yet another group of interesting compounds are those compounds of formula (I) wherein R⁷ is hydrogen; and R⁶ is C₁₋₆alkyl or halo, preferably chloro, especially 4-chloro.

A particular group of compounds are those compounds of formula (I) wherein R⁸ is hydrogen, hydroxy, haloC₁₋₆alkyl, hydroxyC₁₋₆alkyl, cyanoC₁₋₆alkyl, C₁₋₆alkyloxy-carbonylC₁₋₆alkyl, imidazolyl, or a radical of formula —NR¹¹R¹² wherein R¹¹ is hydrogen or C₁₋₁₂alkyl and R¹² is hydrogen, C₁₋₆alkyl, C₁₋₆alkyloxy, hydroxy, C₁₋₆alkyloxyC₁₋₆alkylcarbonyl, or a radical of formula -Alk²-OR¹³ wherein R¹³ is hydrogen or C₁₋₆alkyl.

Preferred compounds are those compounds wherein R¹ is hydrogen, C₁₋₆alkyl, C₁₋₆alkyloxyC₁₋₆alkyl, di(C₁₋₆alkyl)aminoC₁₋₆alkyl, or a radical of formula -Alk¹-C(═O)—R⁹, wherein Alk¹ is methylene and R⁹ is C₁₋₈alkylamino substituted with C₁₋₆alkyloxycarbonyl; R² is halo, C₁₋₆alkyl, C₂₋₆alkenyl, C₁₋₆alkyloxy, trihalomethoxy, hydroxyC₁₋₆alkyloxy or Ar¹; R³ is hydrogen; R⁴ is methyl bound to the nitrogen in 3-position of the imidazole; R⁵ is hydrogen; R⁶ is chloro; R⁷ is hydrogen; R⁸ is hydrogen, hydroxy, haloC₁₋₆alkyl, hydroxyC₁₋₆alkyl, cyanoC₁₋₆alkyl, C₁₋₆alkyloxycarbonylC₁₋₆alkyl, imidazolyl, or a radical of formula —NR¹¹R¹² wherein R¹¹ is hydrogen or C₁₋₁₂alkyl and R¹² is hydrogen, C₁₋₆alkyl, C₁₋₆alkyloxy, C₁₋₆alkyloxyC₁₋₆alkylcarbonyl, or a radical of formula -Alk²-OR¹³ wherein R¹³ is C₁₋₆alkyl; R¹⁷ is hydrogen and R¹⁸ is hydrogen.

Most preferred compounds of formula (I) are
4-(3-chlorophenyl)-6-[(4-chlorophenyl)hydroxy(1-methyl-1H-imidazol-5-yl)methyl]-1-methyl-2(1H)-quinolinone,
6-[amino(4-chlorophenyl)-1-methyl-1H-imidazol-5-ylmethyl]-4-(3-chlorophenyl)-1-methyl-2(1H)-quinolinone;
6-[(4-chlorophenyl)hydroxy(1-methyl-1H-imidazol-5-yl)methyl]-4-(3-ethoxyphenyl)-1-methyl-2(1H)-quinolinone;
6-[(4-chlorophenyl)(1-methyl-1H-imidazol-5-yl)methyl]-4-(3-ethoxyphenyl)-1-methyl-2(1H)-quinolinone monohydrochloride.monohydrate;
6-[amino(4-chlorophenyl)(1-methyl-1H-imidazol-5-yl)methyl]-4-(3-ethoxyphenyl)-1-methyl-2(1H)-quinolinone,
6-amino(4-chlorophenyl)(1-methyl-1H-imidazol-5-yl)methyl]-1-methyl-4-(3-propylphenyl)-2(1H)-quinolinone; a stereoisomeric form thereof or a pharmaceutically acceptable acid or base addition salt; and
(+)-6-[amino(4-chlorophenyl)(1-methyl-1H-imidazol-5-yl)methyl]-4-(3-chloro-phenyl)-1-methyl-2(1H)-quinolinone (Compound 75 in Table 1 of the Experimental part of WO-97/21701); or a pharmaceutically acceptable acid addition salt thereof. The latter compound is especially preferred.

Further preferred embodiments of the present invention include compounds of formula (IX) wherein one or more of the following restrictions apply:
═X¹—X²—X³ is a trivalent radical of formula (x-1), (x-2), (x-3), (x-4) or (x-9) wherein each R⁶ independently is hydrogen, C₁₋₄alkyl, C₁₋₄alkyloxycarbonyl, amino or aryl and R⁷ is hydrogen;
>Y¹—Y²— is a trivalent radical of formula (y-1), (y-2), (y-3), or (y-4) wherein each R⁹ independently is hydrogen, halo, carboxyl, C₁₋₄alkyl or C₁₋₄alkyloxycarbonyl;
r is 0, 1 or 2;
s is 0 or 1;
t is 0;
R¹ is halo, C₁₋₆alkyl or two R¹ substituents ortho to one another on the phenyl ring may independently form together a bivalent radical of formula (a-1);
R² is halo;
R³ is halo or a radical of formula (b-1) or (b-3) wherein
   R¹⁰ is hydrogen or a radical of formula -Alk-OR¹³.
   R¹¹ is hydrogen;
   R¹² is hydrogen, C₁₋₆alkyl, C₁₋₆alkylcarbonyl, hydroxy, C₁₋₆alkyloxy or mono- or di(C₁₋₆alkyl)aminoC₁₋₆alkylcarbonyl;
   Alk is C₁₋₆alkanediyl and R¹³ is hydrogen;
R⁴ is a radical of formula (c-1) or (c-2) wherein
   R¹⁶ is hydrogen, halo or mono- or di(C₁₋₄alkyl)amino;
   R¹⁷ is hydrogen or C₁₋₆alkyl;
aryl is phenyl.

A particular group of compounds consists of those compounds of formula (IX) wherein ═X¹—X²—X³ is a trivalent radical of formula (x-1), (x-2), (x-3), (x-4) or (x-9), >Y1-Y2 is a trivalent radical of formula (y-2), (y-3) or (y-4), r is 0 or 1, s is 1, t is 0, R¹ is halo, C₍₁₋₄₎alkyl or forms a bivalent radical of formula (a-1), R² is halo or C₁₋₄alkyl, R³ is hydrogen or a radical of formula (b-1) or (b-3), R⁴ is a radical of formula (c-1) or (c-2), R⁶ is hydrogen, C₁₋₄alkyl or phenyl, R⁷ is hydrogen, R⁹ is hydrogen or C₁₋₄alkyl, R¹⁰ is hydrogen or -Alk-OR¹³, R¹¹ is hydrogen and R¹² is hydrogen or C₁₋₆alkylcarbonyl and R¹³ is hydrogen;

Preferred compounds are those compounds of formula (IX) wherein ═X¹—X²—X³ is a trivalent radical of formula (x-1) or (x-4), >Y1-Y2 is a trivalent radical of formula (y-4), r is 0 or 1, s is 1, t is 0, R¹ is halo, preferably chloro and most preferably 3-chloro, R² is halo, preferably 4-chloro or 4-fluoro, R³ is hydrogen or a radical of formula (b-1) or (b-3), R⁴ is a radical of formula (c-1) or (c-2), R⁶ is hydrogen, R⁷ is hydrogen, R⁹ is hydrogen, R¹⁰ is hydrogen, R¹¹ is hydrogen and R¹² is hydrogen;

Other preferred compounds are those compounds of formula (IX) wherein ═X¹—X²—X³ is a trivalent radical of formula (x-2), (x-3) or (x-4), >Y1-Y2 is a trivalent radical of formula (y-2), (y-3) or (y-4), r and s are 1, t is 0, R¹ is halo, preferably chloro, and most preferably 3-chloro or R¹ is C₁₋₄alkyl, preferably 3-methyl, R² is halo, preferably chloro, and most preferably 4-chloro, R³ is a radical of formula (b-1) or (b-3), R⁴ is a radical of formula (c-2), R⁶ is C₁₋₄alkyl, R⁹ is hydrogen, R¹⁰ and R¹¹ are hydrogen and R¹² is hydrogen or hydroxy.

The most preferred compounds of formula (IX) are
7-[4-fluorophenyl)(1H-imidazol-1-yl)methyl]-5-phenylimidazo[1,2-a]quinoline;
α-(4-chlorophenyl)-α-(1-methyl-1H-imidazol-5-yl)-5-phenylimidazo[1,2-a]quinoline-7-methanol;
5-(3-chlorophenyl)-α-(4-chlorophenyl)-α-(1-methyl-1H-imidazol-5-yl)-imidazo[1,2-a]quinoline-7-methanol;
5-(3-chlorophenyl)-α-(4-chlorophenyl)-α-(1-methyl-1H-imidazol-5-yl)imidazo[1,2-a]quinoline-7-methanamine;
5-(3-chlorophenyl)-α-(4-chlorophenyl)-α-(1-methyl-1H-imidazol-5-yl)tetrazolo[1,5-a]quinoline-7-methanamine;
5-(3-chlorophenyl)-α-(4-chlorophenyl)-1-methyl-α-(1-methyl-1H-imidazol-5-yl)-1,2,4-triazolo[4,3-a]quinoline-7-methanol;
5-(3-chlorophenyl)-α-(4-chlorophenyl)-α-(1-methyl-1H-imidazol-5-yl)tetrazolo[1,5-a]quinoline-7-methanamine;
5-(3-chlorophenyl)-α-(4-chlorophenyl)-α-(1-methyl-1H-imidazol-5-yl)tetrazolo[1,5-a]quinazoline-7-methanol;
5-(3-chlorophenyl)-α-(4-chlorophenyl)-4,5-dihydro-α-(1-methyl-1H-imidazol-5-yl)tetrazolo[1,5-a]quinazoline-7-methanol;
5-(3-chlorophenyl)-α-(4-chlorophenyl)-α-(1-methyl-1H-imidazol-5-yl)tetrazolo[1,5-a]quinazoline-7-methanamine;
5-(3-chlorophenyl)-α-(4-chlorophenyl)-N-hydroxy-α-(1-methyl-1H-imidazol-5-yl)tetrahydro[1,5-a]quinoline-7-methanamine;
α-(4-chlorophenyl)-α-(1-methyl-1H-imidazol-5-yl)-5-(3-methylphenyl)tetrazolo[1,5-a]quinoline-7-methanamine; the pharmaceutically acceptable acid addition salts and the stereochemically isomeric forms thereof.

5-(3-chlorophenyl)-α-(4-chlorophenyl)-α-(1-methyl-1H-imidazol-5-yl)tetrazolo[1,5-a]quinazoline-7-methanamine, especially the (−) enantiomer, and its pharmaceutically acceptable acid addition salts are especially preferred.

As used in the foregoing definitions and hereinafter halo defines fluoro, chloro, bromo and iodo; C₁₋₆alkyl defines straight and branched chained saturated hydrocarbon radicals having from 1 to 6 carbon atoms such as, for example, methyl, ethyl, propyl, butyl, pentyl, hexyl and the like; C₁₋₈alkyl encompasses the straight and branched chained saturated hydrocarbon radicals as defined in C₁₋₆alkyl as well as the higher homologues thereof containing 7 or 8 carbon atoms such as, for example heptyl or octyl; C₁₋₁₂alkyl again encompasses C₁₋₈alkyl and the higher homologues thereof containing 9 to 12 carbon atoms, such as, for example, nonyl, decyl, undecyl, dodecyl; C₁₋₁₆alkyl again encompasses C₁₋₁₂alkyl and the higher homologues thereof containing 13 to 16 carbon atoms, such as, for example, tridecyl, tetradecyl, pentedecyl and hexadecyl; C₂₋₆alkenyl defines straight and branched chain hydrocarbon radicals containing one double bond and having from 2 to 6 carbon atoms such as, for example, ethenyl, 2-propenyl, 3-butenyl, 2-pentenyl, 3-pentenyl, 3-methyl-2-butenyl, and the like; C₁₋₆alkanediyl defines bivalent straight and branched chained saturated hydrocarbon radicals having from 1 to 6 carbon atoms, such as, for example, methylene, 1,2-ethanediyl, 1,3-propanediyl, 1,4-butanediyl, 1,5-pentanediyl, 1,6-hexanediyl and the branched isomers thereof. The term “C(═O)” refers to a carbonyl group, “S(O)” refers to a sulfoxide and “S(O)₂” to a sulfon. The term “natural amino acid” refers to a natural amino acid that is bound via a covalent amide linkage formed by loss of a molecule of water between the carboxyl group of the amino acid and the amino group of the remainder of the molecule. Examples of natural amino acids are glycine, alanine, valine, leucine, isoleucine, methionine, proline, phenylanaline, tryptophan, serine, threonine, cysteine, tyrosine, asparagine, glutamine, aspartic acid, glutamic acid, lysine, arginine and histidine.

The pharmaceutically acceptable acid or base addition salts as mentioned hereinabove are meant to comprise the therapeutically active non-toxic acid and non-toxic base addition salt forms which the compounds of formulas (I), (II), (III), (IV), (V), (VI), (VII), (VIII) or (IX) are able to form. The compounds of formulas (I), (II), (III), (IV), (V), (VI), (VII), (VIII) or (IX) which have basic properties can be converted in their pharmaceutically acceptable acid addition salts by treating said base form with an appropriate acid. Appropriate acids comprise, for example, inorganic acids such as hydrohalic acids, e.g. hydrochloric or hydrobromic acid; sulfuric; nitric; phosphoric and the like acids; or organic acids such as, for example, acetic, propanoic, hydroxyacetic, lactic, pyruvic, oxalic, malonic, succinic (i.e. butanedioic acid), maleic, fumaric, malic, tartaric, citric, methanesulfonic, ethanesulfonic, benzenesulfonic, p-toluenesulfonic, cyclamic, salicylic, p-aminosalicylic, pamoic and the like acids.

The compounds of formulae (I), (II), (III), (IV), (V), (VI), (VII), (VIII) or (IX) which have acidic properties may be converted in their pharmaceutically acceptable base addition salts by treating said acid form with a suitable organic or inorganic base. Appropriate base salt forms comprise, for example, the ammonium salts, the alkali and earth alkaline metal salts, e.g. the lithium, sodium, potassium, magnesium, calcium salts and the like, salts with organic bases, e.g. the benzathine, N-methyl-D-glucamine, hydrabamine salts, and salts with amino acids such as, for example, arginine, lysine and the like.

The terms acid or base addition salt also comprise the hydrates and the solvent addition forms which the compounds of formulae (I), (II), (III), (IV), (V), (VI), (VII), (VIII) or (IX) are able to form. Examples of such forms are e.g. hydrates, alcoholates and the like.

The term stereochemically isomeric forms of compounds of formulae (I), (II), (III), (IV), (V), (VI), (VII), (VIII) or (IX), as used hereinbefore, defines all possible compounds made up of the same atoms bonded by the same sequence of bonds but having different three-dimensional structures which are not interchangeable, which the compounds of formulae (I), (II), (III), (IV), (V), (VI), (VII), (VIII) or (IX) may possess. Unless otherwise mentioned or indicated, the chemical designation of a compound encompasses the mixture of all possible stereochemically isomeric forms which said compound may possess. Said mixture may contain all diastereomers and/or enantiomers of the basic molecular structure of said compound. All stereochemically isomeric forms of the compounds of formulae (I), (II), (III), (IV), (V), (VI), (VII), (VIII) or (IX) both in pure form or in admixture with each other are intended to be embraced within the scope of the present invention.

Some of the compounds of formulae (I), (II), (III), (IV), (V), (VI), (VII), (VIII) or (IX) may also exist in their tautomeric forms. Such forms although not explicitly indicated in the above formula are intended to be included within the scope of the present invention.

Whenever used hereinafter, the term “compounds of formulae (I), (II), (III), (IV), (V), (VI), (VII), (VIII) or (IX)” is meant to include also the pharmaceutically acceptable acid or base addition salts and all stereoisomeric forms.

A particularly preferred antiestrogen agent for use in accordance with the invention is tamoxifen. Tamoxifen is commercially available for example from AstraZeneca plc under the trade name Nolvadex and may be prepared for example as described in GB Patent Specifications 1064629 and 1354939 or by processes analogous thereto. Other antiestrogen agents include faslodex commercially available for example from AstraZeneca plc under the trade name Fulvestrant, raloxifene commercially available for example from Eli Lilly under the trade name Evista, toremifene commercially available for example from Schering Corporation under the trade name Fareston, and the tamoxifen analog droloxifene. Aromatase inhibitors include letrazole, anastrozole commercially available for example from AstraZeneca plc under the trade name Arimidex, exemestane commercially available for example from Pharmacia & Upjohn under the trade name under the trade name Aromasin, and vorozole.

The present invention also relates to combinations according to the invention for use in medical therapy for example for inhibiting the growth of tumor cells.

The present invention also relates to the use of combinations according to the invention for the preparation of a pharmaceutical composition for inhibiting the growth of tumor cells.

The present invention also relates to a method of inhibiting the growth of tumor cells in a human subject which comprises administering to the subject an effective amount of a combination according to the invention.

This invention further provides a method for inhibiting the abnormal growth of cells, including transformed cells, by administering an effective amount of a combination according to the invention. Abnormal growth of cells refers to cell growth independent of normal regulatory mechanisms (e.g. loss of contact inhibition). This includes the abnormal growth of: (1) tumor cells (tumors) expressing an activated ras oncogene; (2) tumor cells in which the ras protein is activated as a result of oncogenic mutation of another gene; (3) benign and malignant cells of other proliferative diseases in which aberrant ras activation occurs. Furthermore, it has been suggested in literature that ras oncogenes not only contribute to the growth of tumors in vivo by a direct effect on tumor cell growth but also indirectly, i.e. by facilitating tumor-induced angiogenesis (Rak. J. et al, Cancer Research, 55, 4575-4580, 1995). Hence, pharmacologically targeting mutant ras oncogenes could conceivably suppress solid tumor growth in vivo, in part, by inhibiting tumor-induced angiogenesis.

This invention also provides a method for inhibiting tumor growth by administering an effective amount of a combination according to the present invention, to a subject, e.g. a mammal (and more particularly a human) in need of such treatment. In particular, this invention provides a method for inhibiting the growth of tumors expressing an activated ras oncogene by the administration of an effective amount of combination according to the present invention. The present invention is particularly applicable to the treatment of breast cancer including the advanced disease. Examples of other tumors which may be inhibited include, but are not limited to, lung cancer (e.g. adenocarcinoma and including non-small cell lung cancer), pancreatic cancers (e.g. pancreatic carcinoma such as, for example exocrine pancreatic carcinoma), colon cancers (e.g. colorectal carcinomas, such as, for example, colon adenocarcinoma and colon adenoma), hematopoietic tumors of lymphoid lineage (e.g. acute lymphocytic leukemia, B-cell lymphoma, Burkitt's lymphoma), myeloid leukemias (for example, acute myelogenous leukemia (AML)), thyroid follicular cancer, myelodysplastic syndrome (MDS), tumors of mesenchymal origin (e.g. fibrosarcomas and rhabdomyosarcomas), melanomas, teratocarcinomas, neuroblastomas, gliomas, benign tumor of the skin (e.g. keratoacanthomas), kidney carcinoma, ovary carcinoma, bladder carcinoma and epidermal carcinoma.

This invention also provides a method for inhibiting proliferative diseases, both benign and malignant, wherein ras proteins are aberrantly activated as a result of oncogenic mutation in genes, i.e. the ras gene itself is not activated by mutation to an oncogenic mutation to an oncogenic form, with said inhibition being accomplished by the administration of an effective amount of a combination according to the invention, to a subject in need of such a treatment. For example, the benign proliferative disorder neurofibromatosis, or tumors in which ras is activated due to mutation or overexpression of tyrosine kinase oncogenes may be inhibited by the combinations according to the invention.

The antiestrogen agent and the farnesyl transferase inhibitor may be administered simultaneously (e.g. in separate or unitary compositions) or sequentially in either order. In the latter case, the two compounds will be administered within a period and in an amount and manner that is sufficient to ensure that an advantageous or synergistic effect is achieved. It will be appreciated that the preferred method and order of administration and the respective dosage amounts and regimes for each component of the combination will depend on the particular antiestrogen agent and the farnesyl transferase inhibitor being administered, the route of administration of the combination, the particular tumor being treated and the particular host being treated. The optimum method and order of administration and the dosage amounts and regime can be readily determined by those skilled in the art using conventional methods and in view of the information set out herein.

The farnesyl transferase inhibitor is advantageously administered in an effective amount of from 0.0001 mg/kg to 100 mg/kg body weight, and in particular from 0.001 mg/kg to 10 mg/kg body weight. More particularly, for an adult patient, the dosage is conveniently in the range of 50 to 500 mg bid, advantageously 100 to 400 mg bid and particularly 300 mg bid.

The antiestrogen agent is advantageously administered in a dosage of about 1 to 100 mg daily depending on the particular agent and the condition being treated. Tamoxifen is advantageously administered orally in a dosage of 5 to 50 mg, preferably 10 to 20 mg twice a day, continuing the therapy for sufficient time to achieve and maintain a therapeutic effect. Toremifene is advantageously administered orally in a dosage of about 60 mg once a day, continuing the therapy for sufficient time to achieve and maintain a therapeutic effect. Anastrozole is advantageously administered orally in a dosage of about 1 mg once a day. Droloxifene is advantageously administered orally in a dosage of about 20-100 mg once a day. Raloxifene is advantageously administered orally in a dosage of about 60 mg once a day. Exemestane is advantageously administered orally in a dosage of about 25 mg once a day.

It is especially preferred to administer the farnesyl tranferase inhibitor at a dosage of 100 or 200 mg bid for 7, 14, 21 or 28 days with a dosage of the antiestrogen agent in the ranges indicated above.

In view of their useful pharmacological properties, the components of the combinations according to the invention, i.e. the antiestrogen agent and the farnesyl transferase inhibitor may be formulated into various pharmaceutical forms for administration purposes. The components may be formulated separately in individual pharmaceutical compositions or in a unitary pharmaceutical composition containing both components. Farnesyl protein transferase inhibitors can be prepared and formulated into pharmaceutical compositions by methods known in the art and in particular according to the methods described in the published patent specifications mentioned herein and incorporated by reference; for the compounds of formulae (I), (II) and (III) suitable examples can be found in WO-97/21701. Compounds of formulae (IV), (V), and (VI) can be prepared and formulated using methods described in WO 97/16443, compounds of formulae (VII) and (VIII) according to methods described in WO 98/40383 and WO 98/49157 and compounds of formula (IX) according to methods described in WO 00/39082 respectively.

The present invention therefore also relates to a pharmaceutical composition comprising an antiestrogen agent and a farnesyl tranferase inhibitor of formula (I) together with one or more pharmaceutical carriers. To prepare pharmaceutical compositions for use in accordance with the invention, an effective amount of a particular compound, in base or acid addition salt form, as the active ingredient is combined in intimate admixture with a pharmaceutically acceptable carrier, which carrier may take a wide variety of forms depending on the form of preparation desired for administration. These pharmaceutical compositions are desirably in unitary dosage form suitable, preferably, for administration orally, rectally, percutaneously, or by parenteral injection. For example, in preparing the compositions in oral dosage form, any of the usual pharmaceutical media may be employed, such as, for example, water, glycols, oils, alcohols and the like in the case of oral liquid preparations such as suspensions, syrups, elixirs and solutions; or solid carriers such as starches, sugars, kaolin, lubricants, binders, disintegrating agents and the like in the case of powders, pills, capsules and tablets. Because of their ease in administration, tablets and capsules represent the most advantageous oral dosage unit form, in which case solid pharmaceutical carriers are obviously employed. For parenteral compositions, the carrier will usually comprise sterile water, at least in large part, though other ingredients, to aid solubility for example, may be included. Injectable solutions, for example, may be prepared in which the carrier comprises saline solution, glucose solution or a mixture of saline and glucose solution. Injectable suspensions may also be prepared in which case appropriate liquid carriers, suspending agents and the like may be employed. In the compositions suitable for percutaneous administration, the carrier optionally comprises a penetration enhancing agent and/or a suitable wetting agent, optionally combined with suitable additives of any nature in minor proportions, which additives do not cause a significant deleterious effect to the skin. Said additives may facilitate the administration to the skin and/or may be helpful for preparing the desired compositions. These compositions may be administered in various ways, e.g., as a transdermal patch, as a spot-on, as an ointment.

It is especially advantageous to formulate the aforementioned pharmaceutical compositions in dosage unit form for ease of administration and uniformity of dosage. Dosage unit form as used in the specification and claims herein refers to physically discrete units suitable as unitary dosages, each unit containing a predetermined quantity of active ingredient calculated to produce the desired therapeutic effect in association with the required pharmaceutical carrier. Examples of such dosage unit forms are tablets (including scored or coated tablets), capsules, pills, powder packets, wafers, injectable solutions or suspensions, teaspoonfuls, tablespoonfuls and the like, and segregated multiples thereof.

It may be appropriate to administer the required dose of each component of the combination as two, three, four or more sub-doses at appropriate intervals throughout the course of treatment. The sub-doses may be formulated as unit dosage forms, for example, in each case containing independently 0.01 to 500 mg, for example 0.1 to 200 mg and in particular 1 to 100 mg of each active ingredient per unit dosage form.

### Anti-Tumor Activity of a Combination of a Farnesyl Transferase Inhibitor and an Anti-Estrogen Agent

A combination of a farnesyl transferase inhibitor, namely the compound identified as Compound 75 above (R115777), and an anti-estrogen agent, namely tamoxifen (TMX), was tested for anti-tumor activity in comparison with the activity of the individual components of the combination, as described below.

### Mice/Husbandry

Female Nude-Homo NCRNU non-ovarectimized mice 8 weeks of age were fed ad libitum water and an irradiated standard rodent diet. Mice were housed in stable microisolators on a 12 hour light cycle at 21-22° C. and 40-60% humidity.

### Tumors

Mice were inoculated subcutaneously with 1×10⁷ MCF7 human breast carcinoma cells in the flank. Tumors were monitored initially twice a week and then daily as neoplasms reached the desired size, approximately 100 mg. When the carcinomas reached a size between 62-144 mg in calculated tumor weight, the animals were pair matched into the various treatment groups (group mean tumor weights ranged from 83-85 mg).

Estimated tumor weight was calculated using the formula:

$$Tumor Weight ( mg ) = \frac{{w}^{2} \times l}{2}$$

w=width and l=length in mm of a MCF7 tumor

Estrogen pellets (0.36 mg: α-estradiol, 60 day release) were implanted s.c. in the dorsal region of each mouse two days prior to MCF7 cell inoculation. Fresh estrogen pellets were implanted 64 days after the original implant. On Day 1 the estrogen pellets were removed from the two groups administered Tamoxifen (Groups 3 and 5). The pellets were left in place in Groups 1, 2 and 4, and new estrogen pellets were implanted in mice in Groups 1, 2 and 4 on Day 62 of the experiment. The old pellets were not removed from these groups at the time of replacement. The MCF7 breast tumor xenograft requires exogenous estrogen to be supplied to host mice to support the progressive growth of this carcinoma.

### Drugs

The vehicle was 20% beta-cyclodextrin in 0.1N HCl. Beta-cyclodextrin was added slowly to a constantly stirred approximate volume of 0.1 N HCl to yield a 40% beta-cyclodextrin solution. The mixture was covered with foil and stirred until completely dissolved (several hours). The solution was then brought to final volume and filtered (0.2 μm).

R115777 was dissolved in batches sufficient for seven days dosing at a time. R115777 was pulse sonicated for 10 minutes at 4° C., filtered (0.2 μm) and transferred to sterile 15 or 50 ml vials. This solution was further diluted using 20% beta-dextrin in 0.1 N HCl for lower concentration dose groups. Vials were wrapped in foil and stored at 4° C. The dosing volume (0.2 ml/20 g mouse) was weight adjusted.

Tamoxifen was reconstituted in corn oil at 10 mg/ml. Dosing was not body weight adjusted; each mouse received 100 μL of the solution (1 mg/mouse).

### Treatment Plan

MCF-bearing nude mice were pair-matched on Day 1 into five groups of twelve animals each. Tamoxifen was given s.c. at a dose of 1 mg/mouse qd to end. R115777 was administered orally at 100 mg/kg qd to end. The combination therapy group used the same regimens as were employed in the Tamoxifen and R115777 monotherapy groups. A growth control (no treatment group) and a vehicle control group were included in the study. Estrogen pellets were removed from the Tamoxifen monotherapy and the combination therapy groups on Day 1, to avoid antagonizing the Tamoxifen antiestrogen effect

### End Point

The tumor growth inhibition (TGI) endpoint was used in this study to assess the efficacy of the various treatments. The tumor burden endpoint was set at 1.0 g as measured by calliper. TGI values were determined on the last day of the study (Day 5), when all mice were under test, except those that had expired from treatment-related or procedural causes. The mice were euthanized at termination, their MCF7 tumors were excised and weighed, and the TGI values were calculated from the final group mean carcinoma actual weights (excluding those that underwent tumor shrinkage; CRs or PRs).

At excision, tumors smaller than their size on Day 1 were called PRs (partial regressions), and a mouse with no visible carcinoma was termed a CR (complete regression).

Animals recorded as CRs or PRs were not included in the TGI calculations. The following formula was used to calculate the TGI values:

$$\% TGI = [ 1 - ( \frac{Mean Net Tumor {Weight}_{Treated}}{Mean Net Tumor {Weight}_{Control}} ) ] \times 100 \%$$

### Sample Collection

At endpoint, tumors were removed and weighed. At their endpoint, after Day 27, each tumor was cut in half with a scalpel and half was placed in fifteen to twenty volumes of 10% neutral buffered formalin. The other half was snap-frozen in liquid nitrogen and stored at −80° C. At their endpoint, after Day 30, blood was collected from the remaining mice of Groups 3, 4 and 5 by cardiac puncture under CO₂ anesthesia. Serum was recovered, and stored at −80° C. until the end of the study.

### Results

### Summary of Regression Observed in Established MCF-7 Human Breast Tumor Xenografts

| | Treatment: | | |
|---|---|---|---|
| | TMX | R115777 | TMX + |
| | 1 mg/kg | 100 mg/kg | R115777 |
| Regression | 10% | 93% | 94% |
| in Individual | 78% | 100% | 94% |
| Animals* | 94% | | 78% |
| | 44% | | 95% |
| | 59% | | 88% |
| | 31% | | 31% |
| | | | 100% |

*Values indicate the magnitude of tumor regression expressed as % reduction from pretreatment tumor size Untreated control tumors showed no incidence of regression.

These data show that the tested combination unexpectedly increases cytotoxic tumor regression in comparison to the cytostatic effect of the individual components of the combination.

TABLE 1
Protocol Design For The MCF7 Study

| | | Treatment Regimen 1 | | | | Treatment Regimen 2 | | | |
|---|---|---|---|---|---|---|---|---|---|
| Group | n | Agent | mg/kg | Route | Schedule | Agent | mg/kg | Route | Schedule |
| 1 | 12 | Growth Control | | | | | | | |
| 2 | 12 | Vehicle | | po | QD to end | | | | |
| 3 | 12 | Tamoxifen | 1 mg/mouse | sc | Qod to end | | | | |
| 4 | 12 | R115777 | 100 | po | QD to end | | | | |
| 5 | 12 | Tamoxifen | 1 mg/mouse | sc | Qod to end | R115777 | 100 | po | Qd to end |

1-18. (canceled)

19. A combination of tamoxifen and a farnesyl transferase inhibitor wherein the farnesyl transferase inhibitor is (+)-6-[amino(4-chlorophenyl)(1-methyl-1H-imidazol-5-yl)methyl]-4-(3-chloro-phenyl)-1-methyl-2(1H)-quinolinone; or a pharmaceutically acceptable acid addition salt thereof.

20. A method of inhibiting the growth of human breast carcinoma tumor cells for which ras contributes to the growth of said cells, in a human subject which comprises administering to the subject an effective amount of the composition of claim 19.
